(19) United States
(12) Patent Application Publication
(10) Pub. No.: US 2025/0002841 A1
(43) Pub. Date: Jan. 2, 2025

(54) METHOD FOR PRODUCING ALMOND TISSUE FROM CULTURE MEDIUM

(71) Applicant: Almo Tech, Inc., Long Beach, CA (US)

(72) Inventors: Peter Andrew Whitton, Long Beach, CA (US); John R. Munoz, Long Beach, CA (US)

(73) Assignee: Almo Tech, Inc., Long Beach, CA (US)

(21) Appl. No.: 18/216,528

(22) Filed: Jun. 29, 2023

(51) Int. Cl. C12N 5/04

(52) U.S. Cl. CPC C12N 5/04

(57) ABSTRACT

The present invention is a method for culturing almond seed tissue in a laboratory. Gene mapping of the almond is used to incorporate the almond genes that produce the lipid and protein into a yeast. The tissue is grown as a callus that may then be grown in a solid or liquid medium. The medium is used to feed the growing cells with nutrients at the predetermined conditions of light, temperature, and duration. The tissue cultivated in the laboratory converts to lipids, proteins, and flavonoids that make the almond distinct from other seeds.

### BACKGROUND

California produces about 80% of the world's almonds and 100% of the U.S. commercial supply, and almonds are California's number one agricultural export. Almonds grow on trees and have to be harvested from the trees and processed. It takes about four years for an almond tree to bear fruit and closer to seven years to get a full yield from the tree. This means that there are seven years of planning, planting, irrigating, pruning and tending before an almond tree becomes profitable. This situation makes it hard to commit to a crop that takes years of careful irrigation before it produces a single kernel. According to the California Almond Board, it can take as much as three gallons of water to produce each almond grown, making almond farming very precarious in the drier regions where almonds grow best. For these reasons, almond prices are higher compared to similar nuts and there has been a trend toward smaller almond crops despite the popularity of the nuts. The obstacles of the long incubation period and the high water requirements are targets that the present invention seeks to address.

### SUMMARY OF THE INVENTION

The present invention is a method for culturing almond seed tissue in a laboratory to overcome the vast water requirements and the other inefficiencies of growing almonds strictly from a tree. The tissue is grown as a callus that may then be grown in a solid or liquid medium. The medium is used to feed the growing cells with nutrients at the predetermined conditions of light, temperature, and duration. The tissue cultivated in the laboratory converts to lipids, proteins, and flavonoids that make the almond distinct from other seeds. Gene mapping of the almond is used to incorporate the almond genes that produce the lipid and protein into a yeast.

### DESCRIPTION OF THE DRAWINGS

The present invention is best understood when the detailed description of the invention set forth below is read in conjunction with the accompanying drawings listed below.

FIG. 1 is a schematic diagram of a process for growing almond cultures in a medium.

### DETAILED DESCRIPTION OF THE PREFERRED EMBODIMENTS

The invention involves the growing of almond tissue in a controlled laboratory environment to reduce the water requirements and accelerate the production of almond material when compared with a natural growth rate. The method involves using mature almonds from which cells are extracted for cultivation in a selected medium. These cells are prepared and introduced into a solid culture medium and allowed to grow in the culture for a period of four to six weeks. A callus forms during this period that is taken from the solid medium and cultured into a liquid medium. During this second incubation, sterile sugar solution is added to the liquid medium at a constant rate to facilitate tissue growth as well as a production of lipids and proteins.

The addition of the sterile sugar solution must be balanced to match its consumption to optimize growth and maintain the proper balance. The cultured cells are then harvested after second growth period lasting from six to fourteen weeks. The resulting cellular material is then pressed to release the oils and moisture. The oil can be removed from the liquid medium as it separates from the liquid, while the solid matter can filtered and collected to extract its proteins.

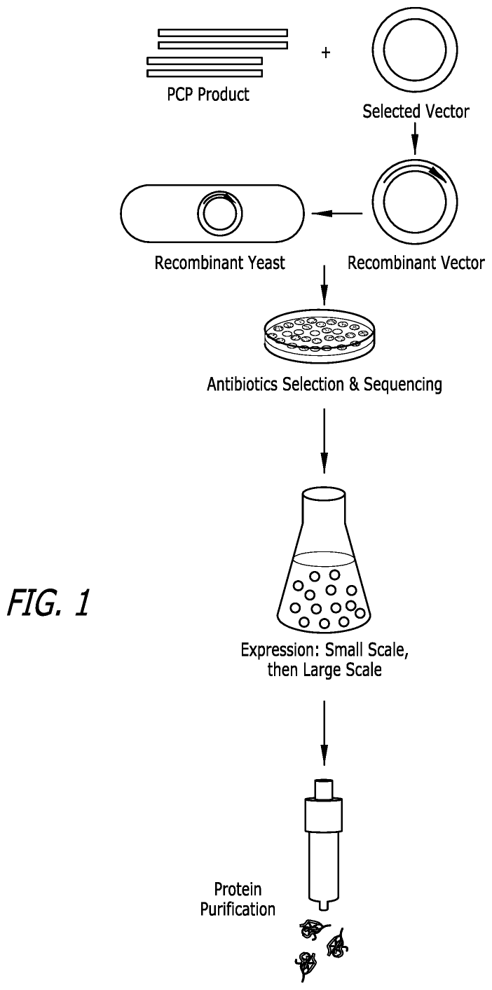

FIG. 1 illustrates the process for growing and extracting the almond products from the culture-grown cells. The first step is taking cells from a mature almond and conducting a gene sequencing to isolate the essential gene(s) needed to grow the almond tissue. To characterize the function of a gene it is necessary to identify the cells in which it is expressed. Traditional methods to determine this include in situ hybridization, gene promoter-reporter fusions or cell isolation/purification techniques followed by quantitative PCR. These methods, although frequently used, can have limitations including their time-consuming nature, limited specificity, reliance upon well-annotated promoters, high cost, and the need for specialized equipment. In situ PCR is a relatively simple and rapid method that involves the amplification of specific mRNA directly within plant tissue whilst incorporating labelled nucleotides that are subsequently detected by immunohistochemistry. In situ polymerase chain reaction (PCR) refers to the amplification of specific nucleic acid sequences and subsequent visualization of the PCR products in tissue sections.

Once the cells have been extracted, the selected vector is introduced as part of the mapping. The most commonly used plant transformation vectors are termed binary vectors because of their ability to replicate in both E. coli, a common lab bacterium and Agrobacterium tumefaciens, a bacterium used to insert the recombinant (customized) DNA into plants. Transformation vercots vectors are linearized plasmids that have been treated to add T overhangs to match the A overhangs of the PCR product. PCR fragments that contain an A overhang can be directly ligated to these T-tailed plasmid vectors with no need for further enzymatic treatment other than the action of T4 DNA ligase.

The resulting recombinant protein is introduced into a solid yeast culture to grow the recombinant proteins that will be the first cultivation step in the process. Once the cellular material is grown in the yeast to a sufficient quantity, the material is transferred to a liquid medium having a sterile sugar infusion at regular intervals to match the consumption of the sugar during the growth stage. This liquid stage growth phase can take between six to fourteen weeks, and the cells form a callus that continues to grow in the nutrient rich medium. Additionally, lipids and proteins are formed in the medium that can be used in subsequent processes.

After the growth of the callus is complete, the material is transferred to a vessel where it can be expressed in multiple stages. The first expression can be used to extract solid materials from the liquid medium, and subsequent expressions can be used to extract oils and proteins to be used in almond products, flavors, and the like. The proteins that are extracted are subjected to a purification process to preserve the essence of the almond proteins and exclude impurities and byproducts.

### Example Methodology

1.1 Cells from the sweet almond, or Prunus dulcis, are obtained from mature nuts so that callus cultures may be grown. Germ tissue of Prunus dulcis seed is sterilized by immersion into a seventy percent ethanol solution for two minutes, followed by immersion into a ten percent bleach solution for ten minutes. The cellular material is then washed three times with sterile (autoclaved) distilled water. The sterile Prunus dulcis seeds have cells removed from the germ in a sterile laminar flow cabinet.

The sterile cultivated cells are introduced into a callus induction media comprised of sterile water with a three percent sucrose and one percent naphthalene acetic acid, and a plant growth medium such as Murashige and Skoog basal powered medium. The prepared media is preferably adjusted to pH of 5.75 and solidified with 0.2% phytagel. The cultured media is then autoclaved for twenty minutes at 121° C. and poured into sterile plastic plant tissue culture dishes.

The cells are then placed onto prepared plates containing callus induction media, and the plates are sealed with a protective film. The sealed plates are then placed into a darkened enclosure at an air temperature of 27° C. In approximately four weeks, a callus formation began to appear.

The medium is prepared by mixing the sucrose, plant growth medium, NAA stock and a 0.01% vitamin stock with distilled water. The constituents are mixed using a magnetic stirrer until all dry components dissolved, then adjust the medium's pH is adjusted with 1M and 0.1M sodium hydroxide (NaOH) to 5.75.

The medium is poured in fifty milliliters quantities into twenty 250 ml conical flasks. The flasks are sealed at the neck with foil. Each sealed flask is then sterilized in an autoclave, at 121° C. 103 kPa, for 25 minutes. Immediately following the sterilization, the flasks are placed into a laminar flow cabinet and allowed to cool to an ambient temperature.

The foil is removed from the prepared media flasks within the sterilized laminar flow cabinet. Thumbnail sized pieces of friable callus from the grown plant tissue are added to the flask until there are approximately five grams of callus for the fifty milliliters of media (10% w/v). The neck of the flasks are then flamed and the flasks are covered with a sterile sheet of foil. The sealed flasks are placed on a shaker set at 120 rpm, in a dark room heated to 27° C. After about two weeks, a thick, dispersed cell suspension culture is observed. The foil is then removed from the flasks within the sterilized laminar flow cabinet.

Using a wide spatula with holes, the cells are scooped out of the media and reintroduced into fresh media at the same ratio of 5 grams of tissue to fifty milliliters of liquid medium. The neck is flamed and a sterile sheet of foil is placed over the mouth of the flask. The flask is placed on a shaker at 120 rpm, in a light room heated to 27° C. After 14 days, the new cell culture are used for further subcultures or harvested and freeze dried.

While a preferred method of forming the cultured material is described above, one of ordinary skill in the art will readily recognize various substitutions and modifications to the inventor's described best mode. The scope of the invention is intended to include all such substitutions and modifications, and should not be limited to any specific step, element, ingredient, or process unless expressly so limited. Rather, the scope of the invention is properly construed using the plain and ordinary meaning of the appended claims, in conjunction with but not limited by the foregoing descriptions and depictions.

1.-8. (canceled)

9. A method for cultivating almond tissue, comprising:
extracting mature almond cells;
performing in situ polymerase chain reaction (PCR) on the almond cells to amplify a nucleic acid sequence of the almond cells;
identifying at least one essential gene needed to grow almond protein based on the PCR step;
introducing transformation vercots vectors to produce a resulting recombinant protein associated with the almond growth protein;
introducing the recombinant protein into a solid yeast culture until a callus is produced;
transferring the callus to a liquid medium and introducing sterile sugar solution at a constant rate equal to a rate of consumption of sugar consumed during a growth stage, said liquid medium stored in an absence of light;
harvesting cultured callus from the liquid medium;
repeating the method using the harvested cultured callus; and
using select callus to extract almond oil and almond protein.
